(19) United States
(12) Patent Application Publication
(10) Pub. No.: US 2012/0238604 A1
(43) Pub. Date: Sep. 20, 2012

(54) CYTOTOXIC COMPOUNDS

(75) Inventor: Robin Scaife, Nedlands (AU)

(73) Assignee: Molecular Discovery Systems, North Perth (AU)

(21) Appl. No.: 13/394,133

(22) PCT Filed: Sep. 3, 2010
(86) PCT No.: PCT/AU10/01144

§ 371 Date: May 16, 2012

(30) Foreign Application Priority Data
Sep. 4, 2009 (AU) 2009904241
Jul. 8, 2010 (AU) 2010903039

(51) Int. Cl. C07C 337/08; C07D 213/72; A61K 31/44; C07D 317/44; A61P 19/00; C07D 249/14; A61K 31/4196; A61P 35/00; A61P 35/02; A61K 31/175; A61K 31/357

(52) U.S. Cl. 514/353; 564/20; 514/583; 546/306; 549/441; 514/466; 548/264.8; 514/383

(57) ABSTRACT

The present invention relates to a compound of general formula (I) or a pharmaceutically acceptable salt or solvate or thereof. In particular, the present invention relates to a method treating diseases involving cell proliferation, migration, apoptosis, or adhesion, comprising administering to a human or non-human mammalian patient an effective amount of a compound of general formula (I) or a pharmaceutically acceptable salt or solvate or thereof.

### FIELD

The present invention relates to novel compounds and compositions which comprise anti-microtubule effects. In particular, the present invention relates to microtubule disrupting compounds, compositions and agents which are useful for the treatment or prophylaxis of proliferative disorders such as cancer, and vasculopathies such as age-related macular degeneration

### BACKGROUND

Hyper-proliferative disorders such as cancer and leukemia affect an estimated 10 million people worldwide. Most treatments are costly and of limited long-term benefit to the affected individuals. As a result, cancer and leukemia continue to be a leading cause of death, and thereby present a substantial socio-economic burden.

Detailed studies of cancerous cells have revealed that unregulated cell proliferation and evasion of cell death (apoptosis) are two of the fundamental hallmarks of cancer. Current options for treating cancers include surgical resection, radiation therapy and/or systemic chemotherapy. Due to various limitations of these treatments, there remains a clear and pressing need for new and/or improved therapies.

Microtubule disrupting agents have proven to be among the most clinically effective anti-cancer drugs. By affecting mitotic spindle function, chromosome segregation during mitosis is perturbed. This generally results in the death of proliferative cell populations, and hyperproliferative cancerous cells are particularly sensitive to drugs that block cell cycle progression and cell division.

Microtubule stabilising drugs (such as Taxol®) directly perturb mitotic spindle function, thereby resulting in the death of cancerous cells and inhibition of tumour growth. Cancerous cells can also be killed by exposure to compounds that destabilise microtubules. While some microtubule destabilising drugs have proven to be too toxic for use as cancer therapeutics (e.g. nocodazole and colchicine), several classes of microtubule destabilising drugs (e.g. combretastatins and indibulins) have been identified that profoundly inhibit tumour growth without causing severe toxic side effects. The anti-tumour activity of combretastatins and several more recently identified microtubule disassembling drugs has been linked to the tumour-selective vascular disruption that occurs in response to disassembly of microtubules in cells of the vascular endothelium. In light of the clinical success of these vascular disrupting agents (VDAs), there is a strong interest in the identification and development of new microtubule disrupting drugs that have similar vascular disruptive and/or tumour inhibitory activities

### SUMMARY

The inventor has identified a new class of compounds that is capable of acting as microtubule disrupting agents. By destabilising microtubules, they inhibit mitotic spindle assembly and/or function(s), and they cause disruption of vascular cell adhesion(s). As a result, they profoundly inhibit the proliferation of human cancer cells, and they can cause inhibition of tumour growth.

Accordingly, in a first aspect, the present invention provides a compound of general formula (I) or a pharmaceutically acceptable salt or solvate thereof:

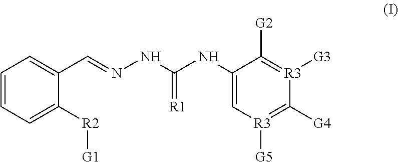

wherein:
R1 is S or O
R2 is O or NH
R3 is C or N
G₁ is H or alkyl

G_{2,3,4,5} is H, halide, alkyl, alkoxy, cyano, amino, hydroxyl or fused phenyl ring and the carbon-nitrogen double bond is preferably in the E conformation.

In some embodiments G₁ is a hydrogen atom or alkyl group of between C₁ and C₁₀. In further embodiments, G₂, G₃, G₄, and/or G₅ is a hydrogen atom, halide (e.g. F, Cl or Br), alkoxy group (e.g. O—CH₂CH₃) short chain alkyl group of between C₁ and C₁₀, preferably containing hetero-atom substitutions such as F (e.g. CF₃), cyano group (e.g. CN), amino group (e.g. NH₂), alkylamino, hydroxyl, or a fused phenyl ring (with or without ring hetero-atoms)

In some embodiments, where a compound or a pharmaceutically acceptable salt thereof is referred to, this phrase may refer to the compound only. In other embodiments, this phrase refers to a pharmaceutically acceptable salt of the compound.

Accordingly, in a first aspect, the present invention provides a compound of general formula (I) or a pharmaceutically acceptable salt or solvate thereof:

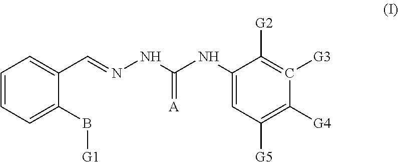

wherein:
A=S or O
B=O or N
C=C or N
G₁=H or alkyl
G_{2,3,4,5}=H, halide, alkyl, alkoxy, cyano, amino, hydroxyl or fused phenyl ring
   and the carbon-nitrogen double bond is preferably in the E conformation.

In some embodiments G₁ is a hydrogen atom or alkyl group (e.g. CH₃). In further embodiments, G₂, G₃, G₄, and/or G₅ is a hydrogen atom, halide (e.g. Cl or Br), alkoxy group (e.g. O—CH2CH3) short chain alkyl group, preferably containing hetero-atom substitutions such as F (e.g. CF3), cyano group (e.g. CN), amino group (e.g. NH2), hydroxyl, or a fused phenyl ring (with or without ring hetero-atoms)

In some embodiments, where a compound or a pharmaceutically acceptable salt thereof, is referred to this refers to the compound only. In another aspect this refers to a pharmaceutically acceptable salt of the compound.

In a second aspect, the present invention provides a compound selected from the group consisting of:

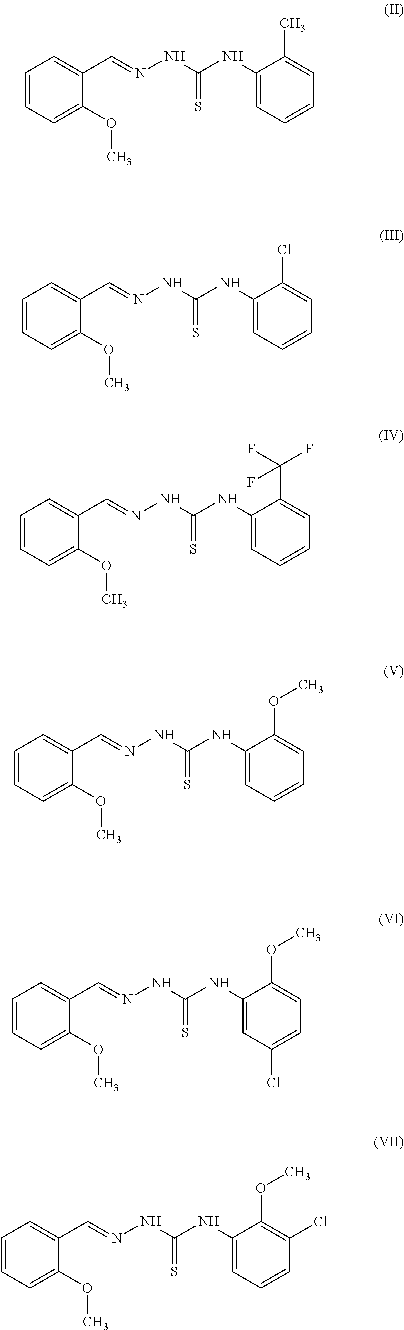

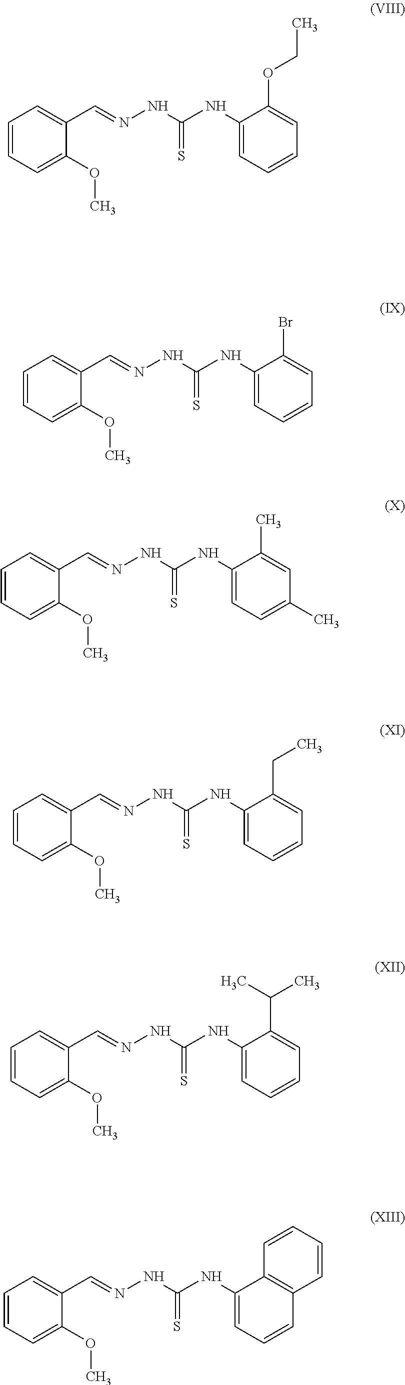

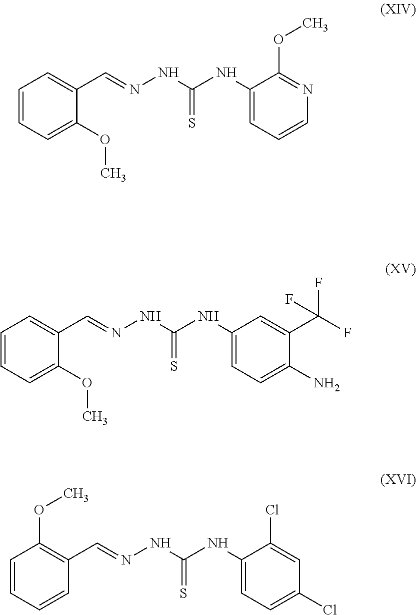

or pharmaceutically acceptable salt or solvate thereof or combination thereof.

In a second aspect, the present invention provides a compound selected from the group consisting of:

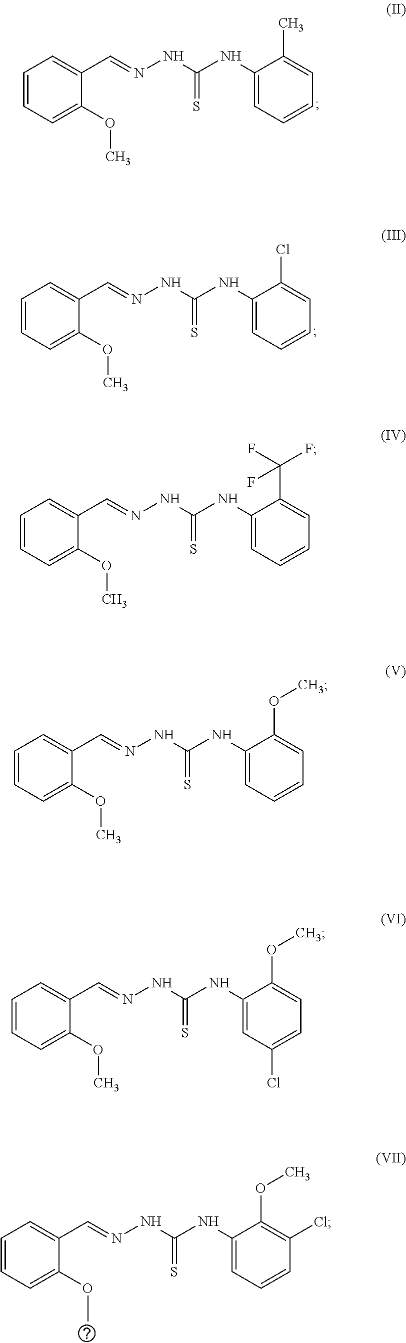

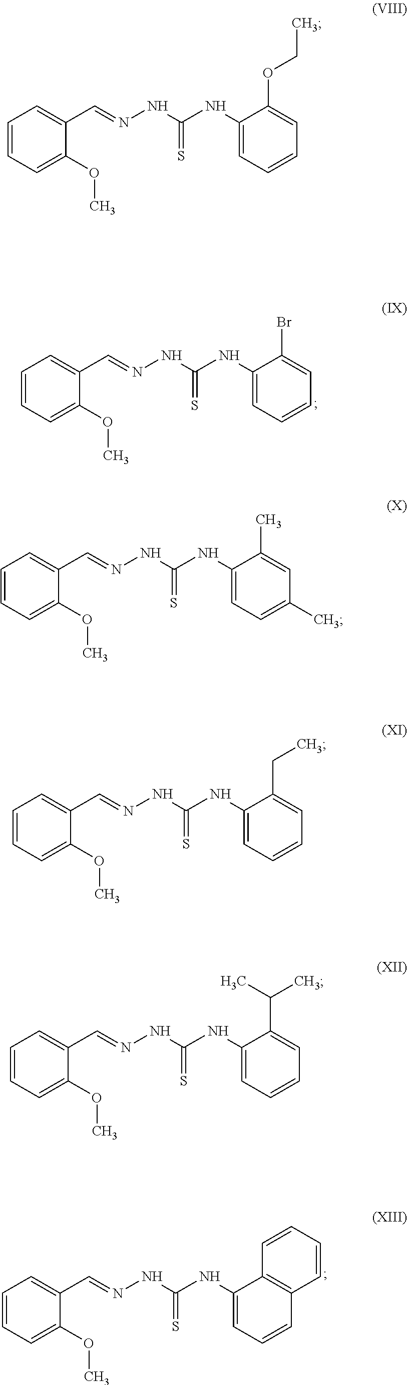

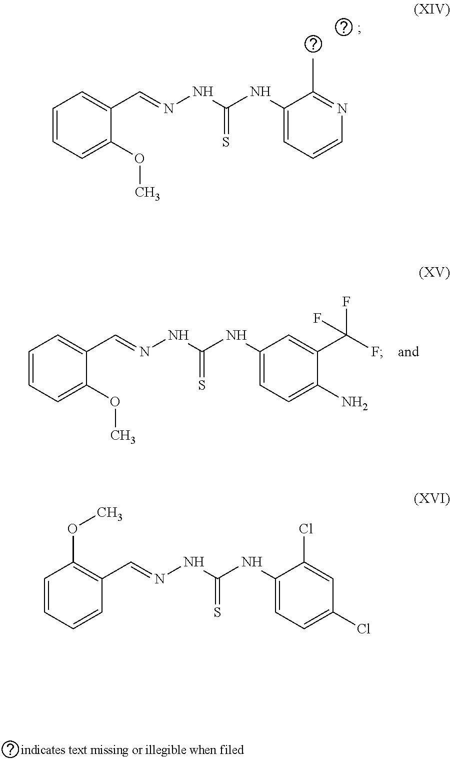

or pharmaceutically acceptable salt or solvate thereof or combination thereof.

In a third aspect, the present invention provides a composition comprising one or more compounds according to the first or second aspects together with a pharmaceutically acceptable carrier.

In a fourth aspect, the present invention provides a method of treating diseases involving cell proliferation, migration, apoptosis, or adhesion comprising administering to a human or non-human mammalian patient an effective amount of a compound according to the first aspect or second aspect or a mixture thereof or a composition according to the third aspect.

In a fifth aspect, the present invention provides a method of treating a proliferative disorder and/or vasculopathy, said method comprising administering to a human or non-human mammalian patient a therapeutically effective amount of a compound according to the first aspect or second aspect or a mixture thereof or a composition according to the third aspect, such that said proliferative disorder is treated.

It will be appreciated by those skilled in the art that any disease involving cell proliferation, migration or apoptosis or any proliferative disorder can be treated with the compounds or compositions of the invention. In some embodiments the disease or disorder is cancer or leukaemia.

As described in detail herein, the compounds and compositions of the invention function by inhibiting microtubule assembly. Accordingly, in a sixth aspect, the present invention provides a method of inhibiting microtubule cytoskeleton function, comprising contacting said microtubules with a compound according to the first aspect or second aspect or a mixture thereof or a composition according to the third aspect, such that said microtubule cytoskeleton function is inhibited.

The compounds and/or compositions of the invention may be presented in kit form. Accordingly, in a seventh aspect, the present invention provides a kit comprising: a) a compound according to the first aspect or second aspect or a mixture thereof or a composition according to the third aspect, in a unit dosage form; and b) a container means for containing said dosage form; and optionally c) with instructions for use.

In an eighth aspect, the present invention provides a use of a compound according to the first aspect or second aspect or a mixture thereof or a composition according to the third aspect for the manufacture of a medicament for treatment of a cancer in a human or non-human mammalian patient.

In a ninth aspect, the present invention provides a compound according to the first aspect or second aspect or a mixture thereof or a composition according to the third aspect for use in the treatment of a cancer in a human or non-human mammalian patient.

### BRIEF DESCRIPTION OF THE FIGURES

FIG. 1. Perturbation of mitotic spindle assembly by compounds A1 and B3.

HeLa cells stably expressing a green fluorescent marker of nuclear DNA (H2B-GFP) were exposed overnight to carrier (DMSO) or 20 μM compounds A1 and B3. The cells were then fixed with methanol, stained with mouse monoclonal anti-β-tubulin antibody and Alexafluor 546 nm goat anti-mouse antibody, and fluorescent signal visualised by microscopy. The open circle indicates a cell with a perturbed mitotic spindle while the arrow indicates a cell with a mostly disassembled mitotic spindle.

FIG. 2. Induction of mitotic arrest by compounds B3 and A1.

(A) HeLa cells were exposed overnight to carrier (DMSO), 20 μM compound B3 or 100 nM paclitaxel, and visualized by phase-contrast microscopy.

(B) HeLa cells exposed for 4 hours to carrier (DMSO), 20 μM compound A1 or 100 nM paclitaxel were fixed and stained with anti-phospho-histone 3 marker of mitosis followed by fluorescent secondary antibody. The percentage of cells positive for phospho-histone 3 was determined by digital imaging and analysis of the fluorescence signals (B).

FIG. 3. Induction of apoptosis by compound A1.

HeLa cells were exposed overnight to 20 μM compound A1 or 100 nM paclitaxel, stained with a fluorescent Annexin V marker of apoptosis and visualised by epifluorescence microscopy following fixation.

FIG. 4. Dose-dependent inhibition of cell proliferation by compound B3. The proliferation of HeLa cells at decreasing concentrations (from 20 to 0.2 μM) of polokinase inhibitor (PKi), KSP inhibitor (STLC) and compound B3 was determined (relative to paclitaxel at 400 to 10 nM) by counting of the cells over time (0-96 h).

FIG. 5 Dose-dependent inhibition of in vitro microtubule assembly by compound C9.

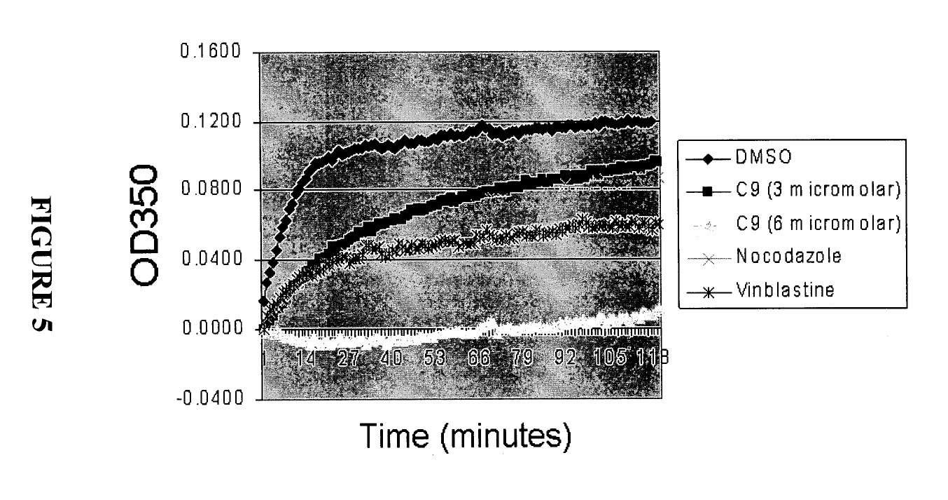

In vitro polymerisation of purified porcine brain tubulin at 37° C. in the presence of compound C9 (3 and 6 μM), 15 μM nocodazole, 2 μM vinblastine or an equal volume of carrier DMSO was measured over time as light scattering at 350 nm.

FIG. 6 Disruption of the microtubule cytoskeleton of vascular endothelium cells by compound C9 Vascular endothelium MS-1 cells on glass coverslips were fixed in 4% p-formaldehyde following exposure to 100 nM compound C9 or an equivalent volume of DMSO carrier for 120 minutes. After staining with Hoescht, TRITC-phalloidin, anti-beta tubulin antibody and Alexafluor 488 nm secondary antibody, the cells were imaged at 400× magnification by epifluorescence microscopy.

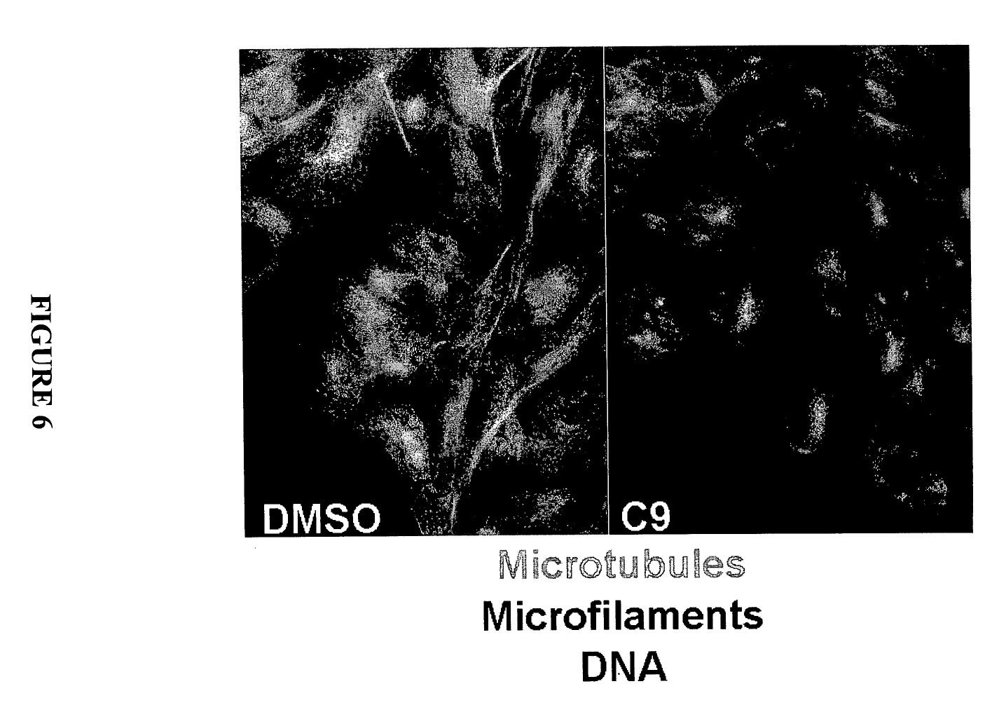

FIG. 7 Disruption of vascular endothelium cell adhesion by compound C9 Vascular endothelium MS-1 cells were allowed to adhere to an extracellular matrix substrate (Matrigel, BD Sciences) for 4 h, resulting in the formation of cell capillaries. Following the addition of 300 nM compound C9 or an equivalent volume of DMSO carrier for 60 minutes, the extracellular matric plugs were imaged at 40× magnification by phase contrast microscopy.

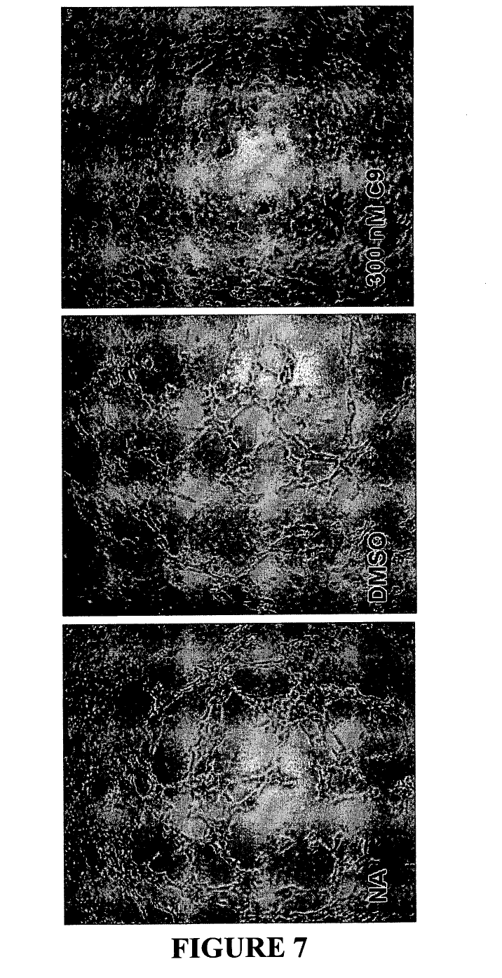

### DESCRIPTION OF THE PREFERRED EMBODIMENTS OF INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional chemistry and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); The Merck Index, 12^{th} Edition (1996), Therapeutic Category and Biological Activity Index; and Remington's Pharmaceutical Sciences, 17^{th} Edition, Mack Publishing Company, Easton, Pa., USA.

It must be noted that as used herein and in the appended claims, the singular forms “a,” “an,” and “the” include plural reference unless the context clearly dictates otherwise. Thus, for example, a reference to “a compound” includes a plurality of such compounds, and a reference to “an analogue” is a reference to one or more analogues, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In its broadest aspect, the present invention provides a compound of general formula (I) or a pharmaceutically acceptable salt or solvate thereof:

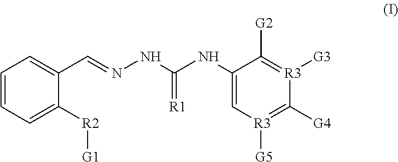

wherein:
R1 is S or O
R2 is O or NH
R3 is C or N
G₁ is H or alkyl
G_{2,3,4,5} is H, halide, alkyl, alkoxy, cyano, amino, hydroxyl or fused phenyl ring and the carbon-nitrogen double bond is preferably in the E conformation.

The term “alkyl” as used herein means an aliphatic hydrocarbon group which may be straight or branched, and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more, lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl. In some embodiments, the alkyl is a short chain alkyl containing halide.

As used herein, the term “halide” denotes a fluoride, chloride, bromide, or iodide.

The term “substituted” means that one or more hydrogen on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent. The term “optionally substituted” means optional substitution with the specified groups, radicals or moieties.

In some embodiments, of the present invention the compound of the present invention is selected from the group consisting of formulas (II) to (XVI):

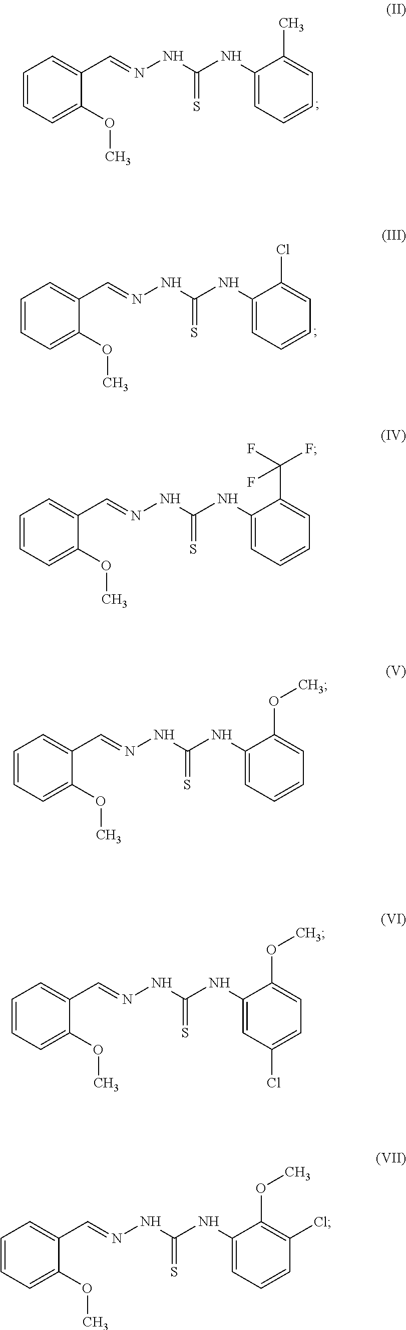

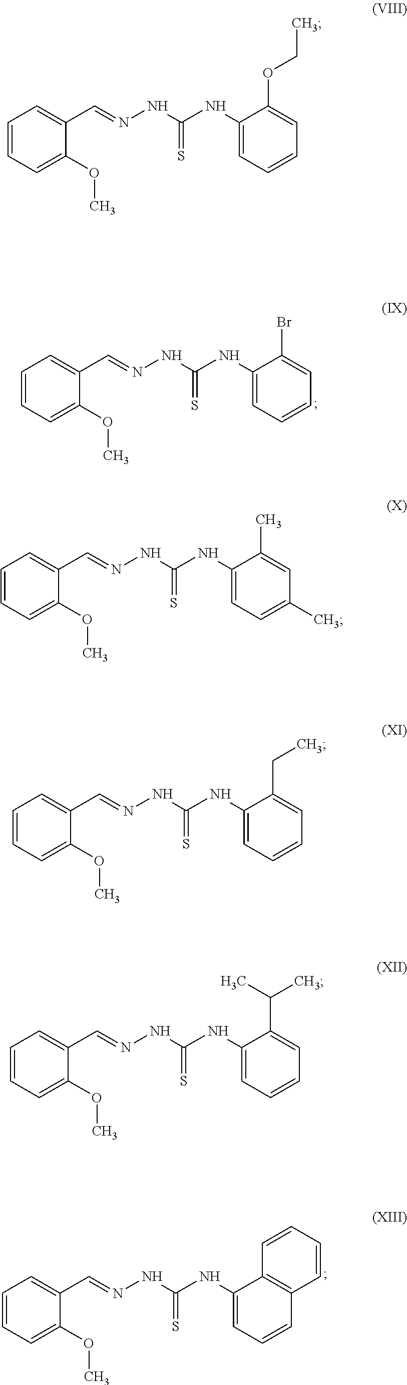

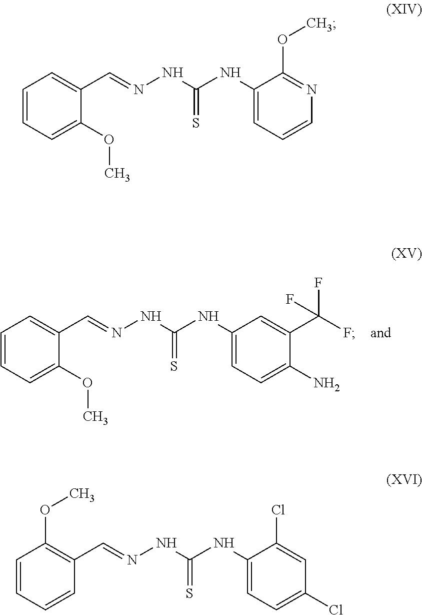

or pharmaceutically acceptable salt or solvate thereof or combination thereof.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of any of Formulas Ito XVI or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

Similarly, if a compound of any of Formulas Ito XVI contains an hydroxyl functional group, a prodrug can be formed by the replacement of the hydrogen atom of the hydroxyl group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)2 or glycosyl (the radical resulting from the removal of a hydroxyl group of the hemiacetal form of a carbohydrate), and the like.

Alternatively, if a compound of any of Formulas Ito XVI contains an amino functional group, a prodrug can be formed by the replacement of the hydrogen atom of the amine group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)2, —P(O)(O(C₁-C₆)alkyl)₂ or glycosyl (the radical resulting from the removal of a hydroxyl group of the hemiacetal form of a carbohydrate), and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci, 93(3), 601-611 (2004) describes the preparation of the solvates of the antifungal fluconazole in ethyl acetate as well as from water. Similar preparations of solvates, hemisolvate, hydrates and the like are described by E. C. van Tonder et al, AAPS PharmSciTech., 5JT), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

The compounds of any of Formulas Ito XVI can form salts which are also within the scope of this invention. Reference to a compound of any of Formulas Ito XVI herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of any of Formulas Ito XVI contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of any of Formulas Ito XVI may be formed, for example, by reacting a compound of any of Formulas Ito XVI with an amount of acid or base, such as an equivalent amount, in a medium such as one in which the salt precipitates or in an aqueous medium followed by lyophilization. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley—VCH; S. Berge et al, Journal of Pharmaceutical Sciences (1977) 66(1^{st}) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The compounds of any of Formulas Ito XVI may contain asymmetric or chiral centers, and, therefore, exist in different stereoisomeric forms. It is intended that all stereoisomeric forms of the compounds of any of Formulas Ito XVI as well as mixtures thereof, including racemic mixtures, form part of the present invention. In addition, the present invention embraces all geometric and positional isomers. For example, if a compound of any of Formulas Ito XVI incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of any of Formulas Ito XVI may be atropisomers (e.g., substituted biaryls) and are considered as part of this invention. Enantiomers can also be separated by use of chiral FPLC column.

It is also possible that the compounds of any of Formulas Ito XVI may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers

The use of the terms “salt”, “solvate”, “prodrug” and the like, is intended to equally apply to the salt, solvate, and prodrug of enantiomers, stereoisomers, rotamers, tautomers, positional isomers, racemates or prodrugs of the inventive compounds.

The synthesis of any of the compounds depicted in Formulas Ito XVI can be undertaken by standard procedures known in the art. Once obtained the compounds are purified.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan

In some embodiments, the compounds of the present invention are combined with other compounds or agents to form compositions. As used herein, the term “composition” is intended to encompass a product comprising the specified ingredients in the specified amounts, as well as any product which results, directly or indirectly, from combination of the specified ingredients in the specified amounts.

In some embodiments, the compounds are combined with pharmaceutically acceptable carriers, diluents or excipients. Pharmaceutically acceptable carriers, diluents or excipients are well known in the art. For example, U.S. Pat. No. 6,689,803, describes several “polymeric carriers”. Representative examples of polymeric carriers include biodegradable compositions such as albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Ilium, L., Davids, S. S. (eds.) “Polymers in Controlled Drug Delivery” Wright, Bristol, 1987; Arshady, J. Controlled Release 17: 1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g. alginate, carrageenin, carboxymethyl cellulose and poly(acrylic acid), or cationic (e.g., chitosan, poly-L-lysine, polyethylenimine, and poly(allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11): 1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly (caprolactone) or poly(lactic acid) with a polyethylene glycol (e.g., PEG), and blends thereof.”

Once produced, the compounds and compositions of the present invention can be used as microtubule disrupting agents or in the treatment of proliferative and vascular disorders.

Herein where the term “microtubule disrupting agent” is used it is to be understood that this refers to compounds of general formula (I), derivatives or chemical analogues thereof or compounds of formula II to XVI, which exert their biological effect by inhibiting or perturbing microtubule assembly, organisation and/or function(s).

Thus, there is provided a method of treating proliferative and vascular disorders such as cancer, in a human or non-human mammalian patient in need of such treatment which comprises administering to said patient an effective amount of compounds I to XVI or a composition thereof as described herein.

The terms “effective amount” or “therapeutically effective amount” is meant to describe an amount of a compound or a composition of the present invention effective in inhibiting proliferative and vascular disorders such as cancer or macular degeneration and thus producing the desired therapeutic, ameliorative, inhibitory or preventative effect.

More specifically, the treatment of the present invention comprises administration of the compounds of any of Formulas Ito XVI to a patient for the treatment of a variety of cancers or vasculopathies, including (but not limited to) the following: tumour of the bladder, breast (including BRCA-mutated breast cancer), colorectal, colon, kidney, liver, lung (including small cell lung cancer and non-small cell lung cancer), head and neck, oesophagus, bladder, gall bladder, ovary, pancreas, stomach, cervix, thyroid, prostate, and skin, including squamous cell carcinoma; leukaemia, acute lymphocytic leukaemia, acute lymphoblastic leukaemia, B-cell lymphoma, T-cell lymphoma, Hodgkins lymphoma, non-Hodgkins lymphoma, hairy cell lymphoma, mantle cell lymphoma, myeloma and Burkett's lymphoma; chronic lymphocytic leukaemia (“CLL”), acute and chronic myelogenous leukaemia, myelodysplastic syndrome and promyelocytic leukemia; fibrosarcoma, rhabdomyosarcoma; mantle cell lymphoma, myeloma; astrocytoma, neuroblastoma, glioblastoma, malignant glial tumors, hepatocellular carcinoma, gastrointestinal stromal tumors (“GIST”) and schwannomas; melanoma, multiple myeloma, seminoma, teratocarcinoma, osteosarcoma, xenoderoma pigmentosum, keratoctanthoma, thyroid follicular cancer and Kaposi's sarcoma.

In another embodiment of the invention, the cancer is in a metastatic state, and more particularly the cancer produces metastases to the bone. In a further embodiment of the invention, particularly the cancer is in a metastatic state, and more particularly the cancer produces skin metastases. In a further embodiment of the invention, particularly the cancer is in a metastatic state, and more particularly the cancer produces lymphatic metastases. In a further embodiment of the invention, the cancer is in a non-metastatic state.

For the avoidance of doubt, where the treatment of cancer is indicated, it is to be understood that this also refers to the prevention of metastases and the treatment of metastases, i.e. cancer spread. Therefore the combination of the present invention could be used to treat a patient who has no metastases to stop them occurring, or to lengthen the time period before they occur, and to a patient who already has metastases to treat the metastases themselves. Furthermore the treatment of cancer also refers to treatment of an established primary tumour or tumours and developing primary tumour or tumours. In one aspect of the invention the treatment of cancer relates to the prevention of metastases. In another aspect of the invention the treatment of cancer relates to the treatment of metastases. In another aspect of the invention the treatment of cancer relates to treatment of an established primary tumour or tumours or developing primary tumour or tumours. Herein, the treatment of cancer also refers to the prevention of cancer per se.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally or intravenously.

Also contemplated are delivery methods that are combinations of the above-noted delivery methods. Such methods are typically decided by those skilled in the art.

Preferably, the pharmaceutical preparation is in a unit dosage form. In such form, the preparation is subdivided into suitably sized unit doses containing appropriate quantities of the active components.

Another aspect of this invention is a kit comprising a therapeutically effective amount of at least one compound of any of Formulas Ito XVI, or a pharmaceutically acceptable salt, solvate, or prodrug of said compound and a pharmaceutically acceptable carrier, vehicle or diluent.

In some embodiments, the compounds and compositions of the present invention are combined with other therapeutic agents including other anti-mitotic agents or cytotoxic agents. Therefore according to the present invention, there is provided a combination, comprising one or more of compounds of formula Ito XVI in combination with an anti-mitotic or cytotoxic agent for use as a medicament.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial and/or synergistic effect of the combination.

According to a further aspect of the invention there is provided a pharmaceutical composition which comprises one or more of compounds of formula Ito XVI in combination with a cytotoxic agent in association with a pharmaceutically acceptable diluent or carrier.

According to a further aspect of the present invention there is provided a kit comprising a compound of general formula (I); optionally with instructions for use.

According to a further aspect of the present invention there is provided a kit comprising:

a) a compound of general formula (I), in a unit dosage form;
b) container means for containing said unit dosage form; and optionally
c) with instructions for use.

An example of a unit dosage from for a compound of general formula (I) might be a tablet for oral formulation, see that described herein below.

The pharmaceutical compositions may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. In general the above compositions may be prepared in a conventional manner using conventional excipients.

For example, a compound of general formula (I) can be formulated as a tablet using the following excipients:

### Compound (I);

Lactose monohydrate (filler);
Croscarmellose sodium (disintegrant);
Povidone (binder);
Magnesium stearate (lubricant);
Hypromellose (film coat component);
Polyethylene glycol 300 (film coat component); and
Titanium dioxide (film coat component).

The amount of a compound of general formula (I), or a pharmaceutically acceptable salt thereof, administered would be that sufficient to provide the desired pharmaceutical effect. For instance, compound (I) could be administered to a warm-blooded animal orally, at a unit dose less than 1 g daily but more than 2.5 mg. Particularly compound (I) could be administered to a warm-blooded animal, at a unit dose of less than 250 mg per day. In another aspect of the invention, compound (I) could be administered to a warm-blooded animal, at a unit dose of less than 130 mg per day. In a further aspect of the invention, compound (I) could be administered to a warm-blooded animal, at a unit dose of less than 50 mg per day.

The dosage of each of the drugs and their proportions have to be composed so that the best possible treatment effects, as defined by national and international guidelines (which are periodically reviewed and re-defined), will be met.

The present invention further provides use of a compound or composition of the present invention for the manufacture of a medicament for treatment of a cancer in a human or non-human mammalian patient.

Although preferred embodiments have been depicted and described in detail herein, it will be apparent to those skilled in the relevant art that various modifications, additions, substitutions, and the like can be made without departing from the spirit of the invention, and these are thus considered to be within the scope of the invention as defined in the claims which follow.

Throughout the specification, the word “comprise” and variations of the word, such as “comprising” and “comprises,” means “including but not limited to” and is not intended to exclude other additives, components, integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

Thus, the phrase “consisting essentially of” indicates that the listed elements are required or mandatory, but that no other elements are optional and may or may not be present depending upon whether or not they affect the activity or action of the listed elements.

The invention will now be further described by way of reference only to the following non-limiting examples. It should be understood, however, that the examples following are illustrative only, and should not be taken in any way as a restriction on the generality of the invention described above. In particular, while the invention is described in detail in relation to 46 compounds shown in Examples 1 to 6 it will be clearly understood that the findings herein are not limited to these compounds.

### Example 1

### Perturbation of Mitotic Spindle Organisation and Assembly by Compounds A1 and B3

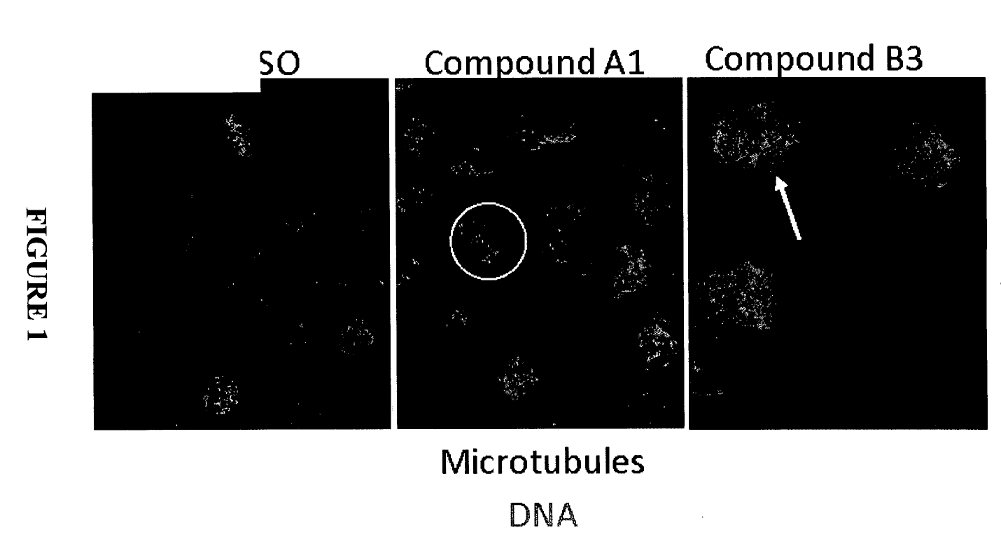

As shown in FIG. 1, HeLa cells expressing a green fluorescent marker of nuclear DNA (generated by stable expression of the histone 2B cDNA fused to green-fluorescent protein (GFP) cDNA) were propagated in DMEM growth medium supplemented with 10% (v/v) fetal bovine serum. These cells were exposed overnight to carrier (0.1% (v/v) DMSO) or an equivalent volume of DMSO containing compounds A1 and B3 (final concentration in the culture medium was 20 μM). The cells were then briefly rinsed with phosphate-buffered saline (PBS) prior to fixation with 100% methanol at −20° C. The fixed cells were then stained with mouse monoclonal anti-β-tubulin antibody and Alexafluor 546 nm goat anti-mouse antibody (diluted 400× from commercial stocks into PBS containing 10 mg/ml bovine serum albumin (BSA)). The GFP and 546 nm fluorescence signals were then visualised using a microscope equipped for digital confocal epifluorescence imaging. The open circle in FIG. 1 represents a cell with a perturbed mitotic spindle while the arrow points to a cell with a mostly unassembled mitotic spindle.

This indicates that prolonged exposure of these cells to compound A1 results in substantial alterations in the spatial organisation of mitotic spindle fibres and chromosomes, while exposure to compound B3 profoundly perturbs spindle function by blocking the assembly of spindle microtubule fibres.

### Example 2

### Induction of Mitotic Arrest by Compounds A1 and B3

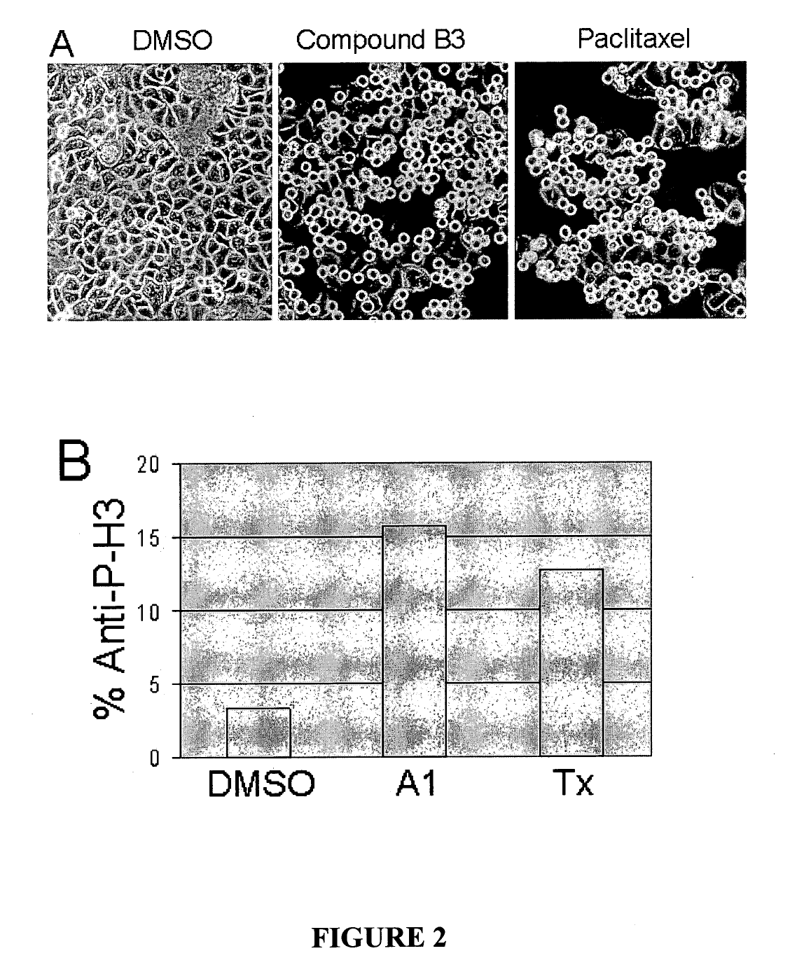

FIG. 2 (A) The H2B-GFP-expressing HeLa cells (grown under identical culture conditions as described in Example 1) were exposed overnight to carrier (0.1% (v/v) DMSO), 20 μM compound B3 or 100 nM paclitaxel in DMSO. The cells were then visualized using a phase-contrast microscope equipped for digital imaging.

It can be seen that, similar to the effect of paclitaxel, exposure of these cells to compound B3 results in a pronounced accumulation of round and translucent cells representing the mitotic phenotype.

FIG. 2 (B) The H2B-GFP-expressing HeLa cells were treated for 4 hours with carrier (0.1% (v/v) DMSO), 20 μM compound A1 or 100 nM paclitaxel in DMSO. The cells were then briefly rinsed with phosphate-buffered saline (PBS) prior to fixation with 4% para-formaldehyde at ambient temperature. The fixed cells were then stained with mouse monoclonal anti-phospho-histone 3 antibody and Alexafluor 546 nm goat anti-mouse antibody (diluted 400× from commercial stocks into PBS containing 10 mg/ml bovine serum albumin (BSA)). The GFP and 546 nm fluorescence signals were then captured with an IN Cell Analyser 1000 (GE Healthcare) and the percentage of cells stained with the anti-phospho-histone 3 antibody determined using Developer™ software (GE Healthcare).

It can be seen that, similar to the effect of paclitaxel, exposure of these cells to compound B3 results in the accumulation of cells that are stained with the phospho-histone 3 marker of mitosis.

### Example 3

### Induction of Apoptosis by Compound A1

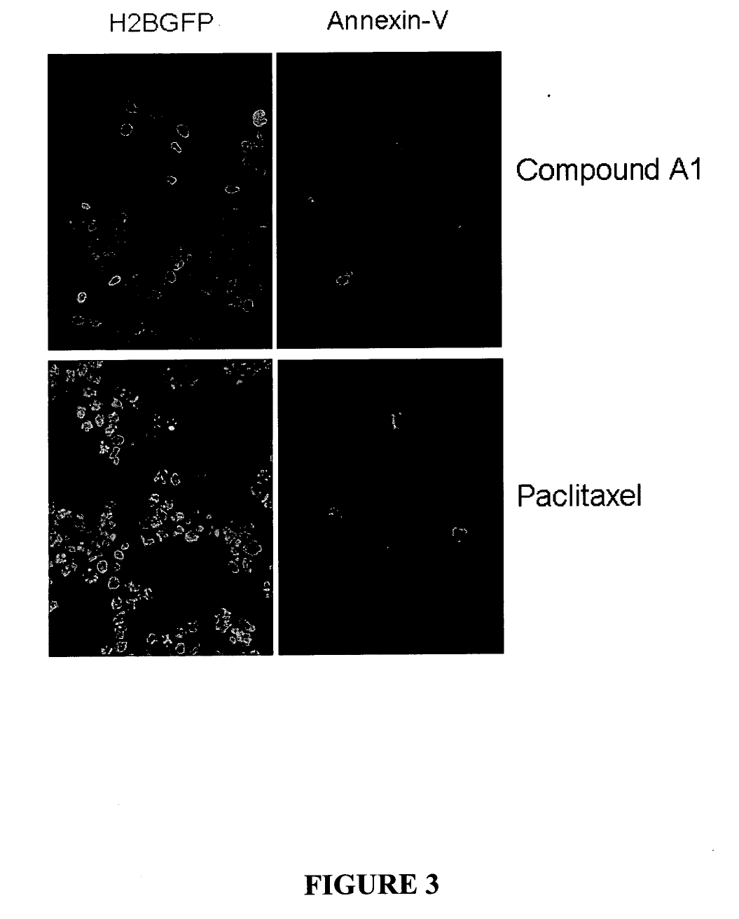

FIG. 3 The H2B-GFP-expressing HeLa cells were treated overnight with 20 μM compound A1 or 100 nM paclitaxel. The cells were then exposed to Annexin-V tagged with Alexafluor 546 nm and visualized using an epifluorescence microscope equipped for digital imaging following fixation with 4% para-formaldehyde.

It can be seen that, similar to the effect of paclitaxel, exposure of these cells to compound A 1 results in the induction of apoptotic cells identified by the binding of Annexin-V.

### Example 4

### Dose-Dependent Inhibition of Cell Proliferation by Compound B3

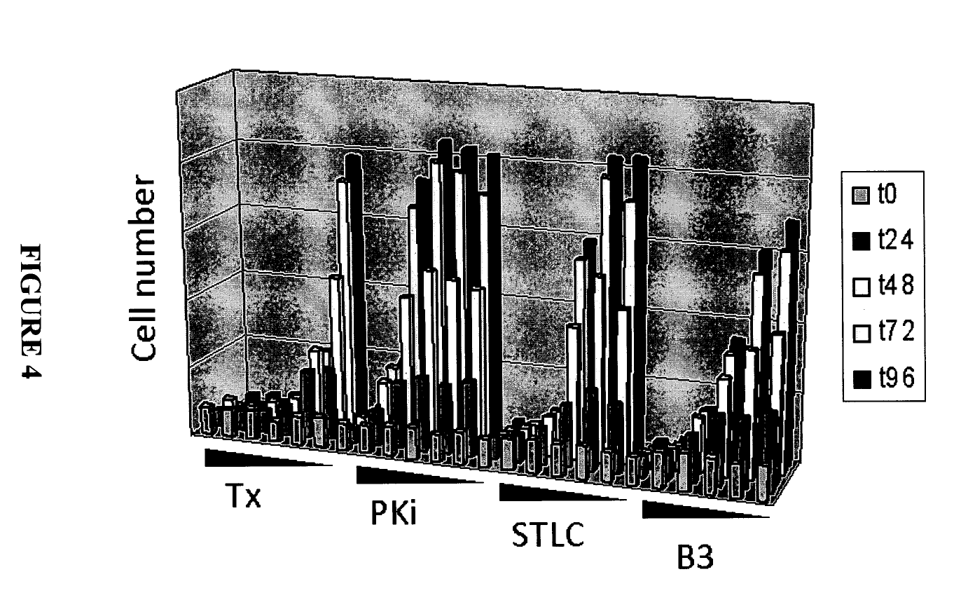

FIG. 4 HeLa cells expressing H2B-GFP were plated in multi-well plates and exposed to 0.2-20 μM of polokinase inhibitor (PKi), KSP inhibitor (STLC) and compound B3, or 10-400 nM of paclitaxel. The number of cells at 0, 24, 48, 72 and 96 h of treatment was determined by imaging of the GFP signal in the plates with an IN Cell Analyser (GE Healthcare) followed by measurement of the number of intact cell nuclei using Developer™ software (GE Healthcare).

It can be seen that compound B3 inhibits the proliferation of these cells in a dose-dependent manner, potently blocking the proliferation at concentrations in excess of 1 μM.

### Example 5

Table 1 HeLa cells expressing H2B-GFP were plated in multi-well plates and exposed overnight to 20 μM of the indicated compounds A1-A10. The relative number of mitotic cells was then determined by imaging of the plates using phase-contrast microscopy.

It can be seen that introduction of the 2-methoxy group (compound A3) increases the inhibition of mitosis by benzaldehyde phenylsemicarbazones

TABLE 1
Relative Anti-mitotic activity

| | | |
|---|---|---|
| A | 2-HYDROXYBENZALDEHYDE N- | 1 |
| | (4-FLUOROPHENYL)THIOSEMICARBAZONE | |
| | 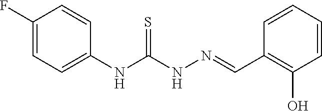 | |

Phenylthiosemicarbazone analogues

| | | |
|---|---|---|
| A1 | BENZALDEHYDE N- | 0 |
| | (4-FLUOROPHENYL)THIOSEMICARBAZONE | |
| | 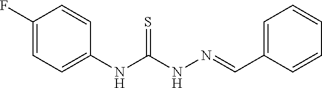 | |
| A2 | 4-HYDROXYBENZALDEHYDE N- | 0 |
| | (4-FLUOROPHENYL)THIOSEMICARBAZONE | |
| | 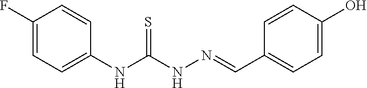 | |
| A3 | 2-METHOXYBENZALDEHYDE N- | 3 |
| | (4-FLUOROPHENYL)THIOSEMICARBAZONE | |
| | 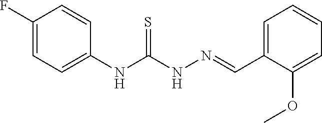 | |
| A4 | 2-HYDROXY-1-NAPHTHALDEHYDE N-(4- | 0 |
| | FLUOROPHENYL)THIOSEMICARBAZONE | |
| | 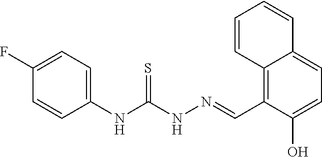 | |
| A5 | 1-NAPHTHALDEHYDE N-(4- | 0 |
| | FLUOROPHENYL)THIOSEMICARBAZONE | |
| | 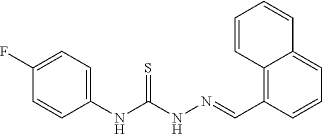 | |
| A6 | 2-Hydroxybenzaldehyde N-ethylthiosemicarbazone | 0 |
| | 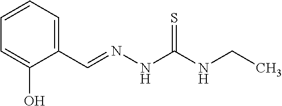 | |
| A7 | 2-HYDROXYBENZALDEHYDE | 1 |
| | N-PHENYLSEMICARBAZONE | |
| | 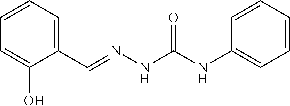 | |
| A8 | 2-HYDROXYBENZALDEHYDE N- | 1 |
| | (3-CHLOROPHENYL)THIOSEMICARBAZONE | |
| | 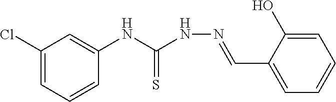 | |
| A9 | 2-Hydroxybenzaldehyde phenylhydrazone | 0 |
| | 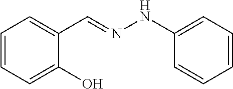 | |
| A10 | Diphenylthiocarbazone | 0 |
| | 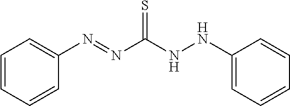 | |

### Example 6

Table 2 HeLa cells expressing H2B-GFP were plated in multi-well plates and exposed overnight to 20 μM of the indicated compounds B1-B9. The relative number of mitotic cells was then determined by imaging of the plates using phase contrast microscopy.

It can be seen that introduction of the 3-methyl (compound B3) and 3-chloro (compound B9) substitutions increases the anti-mitotic activity of benzaldehyde phenylsemicarbazones.

TABLE 2
Relative anti-mitotic activity
Methoxybenzaldehydes

| | | |
|---|---|---|
| B1 | 2-METHOXYBENZALDEHYDE | 0 |
| | THIOSEMICARBAZONE | |
| | 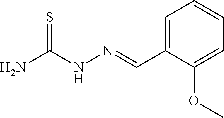 | |
| B2 | 2-METHOXYBENZALDEHYDE N- | 1 |
| | CYCLOHEXYLTHIOSEMICARBAZONE | |
| | 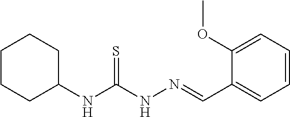 | |
| B3 | 2-METHOXYBENZALDEHYDE N- | 3 |
| | (3-METHYLPHENYL)THIOSEMICARBAZONE | |
| | 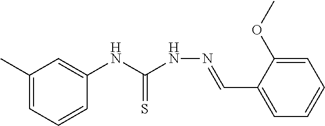 | |
| B4 | 5-BROMO-2-METHOXYBENZALDEHYDE N- | 0 |
| | (4-FLUOROPHENYL)THIOSEMICARBAZONE | |
| | 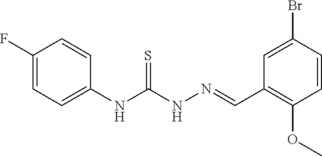 | |
| B5 | 2-HYDROXY-3-METHOXYBENZALDEHYDE N- | 0 |
| | (4-FLUOROPHENYL)THIOSEMICARBAZONE | |
| | 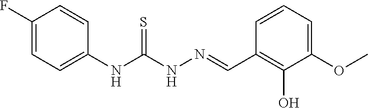 | |
| B6 | 2-HYDROXY-5-METHOXYBENZALDEHYDE N- | 0 |
| | (4-FLUOROPHENYL)THIOSEMICARBAZONE | |
| | 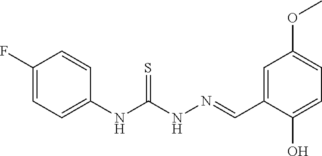 | |
| B7 | 2-ETHOXYBENZALDEHYDE N-(3- | 0 |
| | METHYLPHENYL)THIOSEMICARBAZONE | |
| | 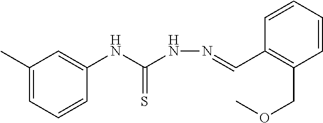 | |
| B8 | 2-METHOXYBENZALDEHYDE N- | 2 |
| | PHENYLTHIOSEMICARBAZONE | |
| | 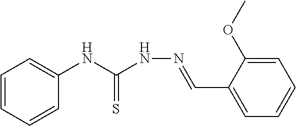 | |
| B9 | 2-METHOXYBENZALDEHYDE N-(3- | 3 |
| | CHLOROPHENYL)THIOSEMICARBAZONE | |
| | 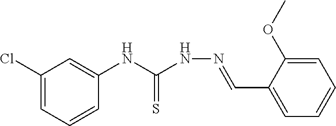 | |

### Example 7

Table 3 HeLa cells expressing H2B-GFP were plated in 96-well plates and exposed to concentrations ranging from 100 μM to 30 μM of the indicated compounds C1-C78. The number of cells was then determined every 24 hours by imaging of the plates using an IN Cell Analyser (GE Healthcare), followed by measurement of the number of intact cell nuclei using Developer™ software (GE Healthcare). Drug concentrations that cause 50% inhibition of cell proliferation (GI50) were determined from the nuclear counts using GraphPad Prism software or ED50v10 freeware.

It can be seen that 2-trifluoromethyl (compound C9), 2-methoxy (compound C15), 2-ethoxy (compound C34), 2,4 dimethyl (compound C42), 2-ethyl (compound C45), 2-isopropyl (compound C46) and 2,4 dichloro (compound C75) substitutions greatly increase the anti-proliferative activity of the compounds.

Parallel plates were treated overnight with the compounds to visualize the mitotic cells by staining with anti-phospho-histone 3 and Alexafluor 546 nm secondary antibodies after fixation with 4% paraformaldehyde. Following imaging of the nuclear GFP and phospho-histone 3 signals using an IN Cell Analyser (GE Healthcare), determination of the mitotic index by analysis of the images with Developer™ software (GE Healthcare) permitted estimation of the concentration that leads to 50% induction of the mitotic arrest (EC50). It can be seen that inhibition of cell proliferation and mitosis correlate well for the compounds tested.

TABLE 3

| | | | EC50 |
|---|---|---|---|
| | | GI50 | (mitosis) |
| | | (HeLa) | (HeLa) |
| C1 | 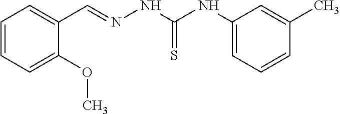 | 700 nM | ≈1 μM |
| C2 | 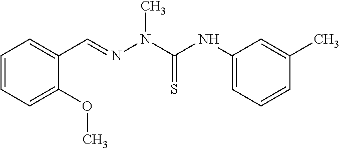 | ≈15,000 nM | |
| C3 | 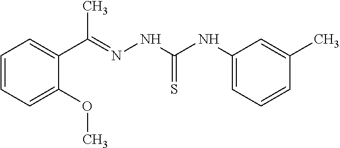 | ≧20,000 nM | |
| C4 | 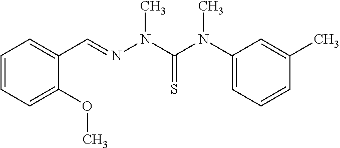 | ≧20,000 nM | |
| C5 | 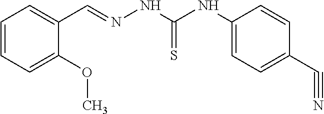 | ≈20,000 nM | |
| C6 | 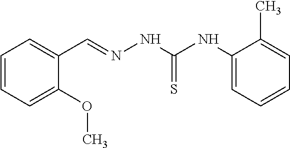 | 90 nM | 150 nM |
| C7 | 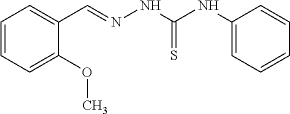 | 5,000 nM | |
| C8 | 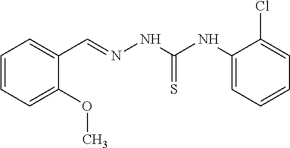 | 100 nM | 300 nM |
| C9 | 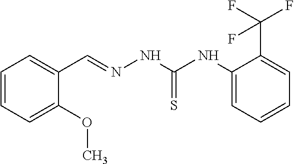 | 10 nM | 10 nM |
| C10 | 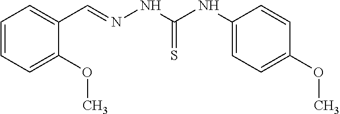 | 2,000 nM | >1 μM |
| C11 | 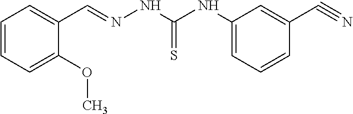 | 2,000 nM | |
| C12 | 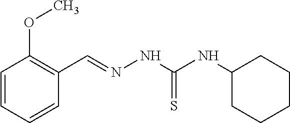 | ≧20,000 nM | |
| C13 | 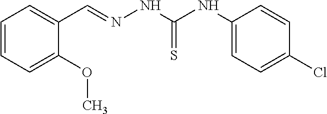 | 1,000 nM | |
| C14 | 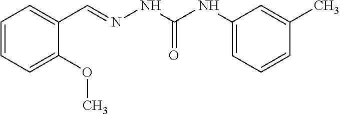 | 4,000 nM | |
| C15 | 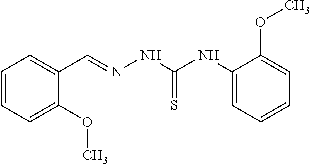 | 25 nM | ≈70 nM |
| C16 | 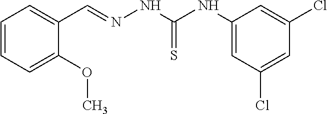 | 2,600 nM | |
| C17 | 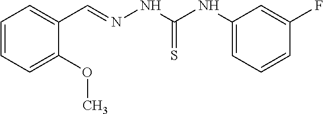 | ≈1,000 nM | |
| C18 | 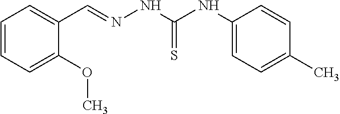 | 700 nM | ≧30 nM |
| C19 | 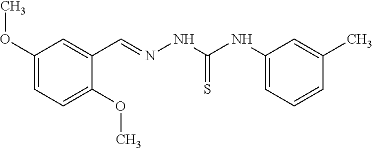 | ≧15,000 nM | |
| C20 | 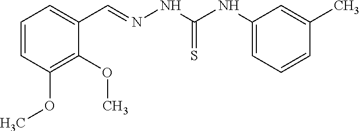 | no activity | |
| C21 | 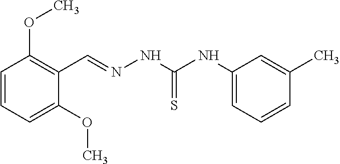 | no activity | |
| C22 | 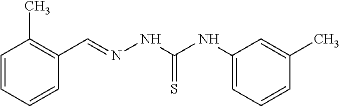 | no activity | |
| C23 | 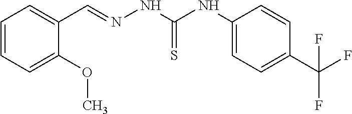 | ≧20,000 nM | |
| C24 | 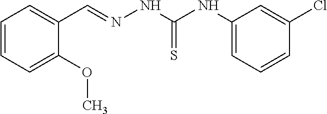 | 500 nM | |
| C25 | 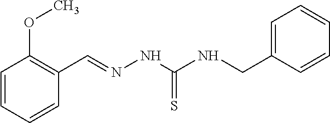 | no activity | |
| C26 | 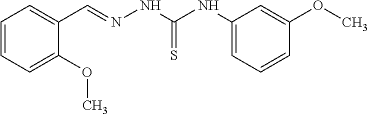 | 1,300 nM | ≈1 μM |
| C27 | 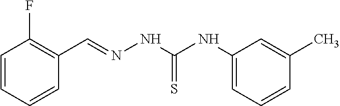 | ≧20,000 nM | |
| C28 | 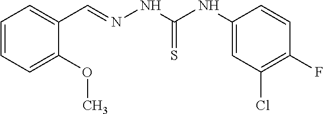 | approx 3,000 nM | |
| CB29 | 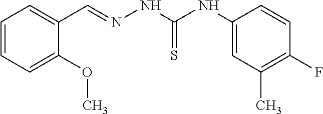 | 1,000 nM | |
| CB30 | 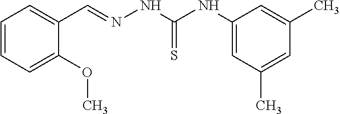 | 500 nM | |
| CB31 | 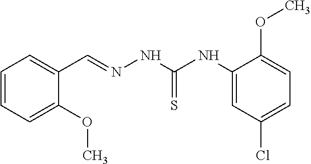 | 15 nM | 50 nM |
| CB32 | 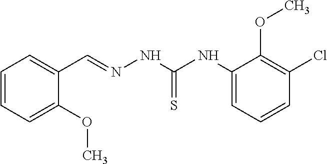 | 25 nM | |
| CB33 | 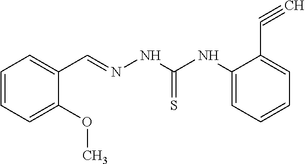 | 1,000 nM | |
| CB34 | 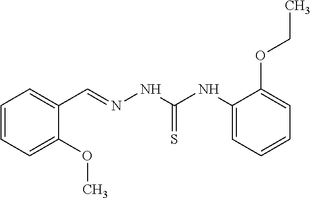 | 11 nM | 10 nM |
| CB35 | 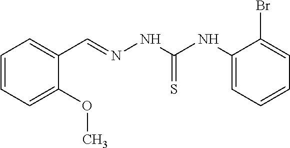 | 50 nM | 100 nM |
| CB36 | 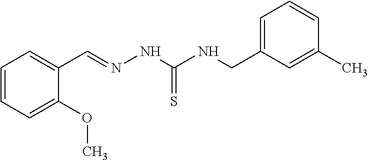 | approx 5,000 nM | |
| CB37 | 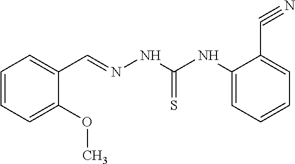 | approx 3,000 nM | |
| CB38 | 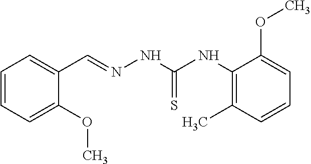 | approx 3,000 nM | |
| CB39 | 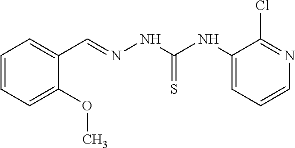 | approx 3,000 nM | |
| CB40 | 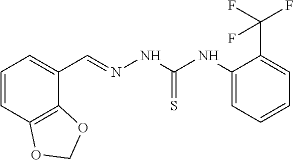 | 500 nM | |
| CB41 | 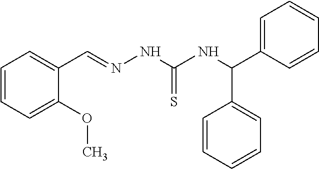 | approx 5,000 nM | |
| CB42 | 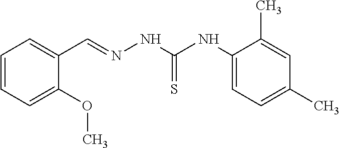 | 9.5 nM | |
| CB43 | 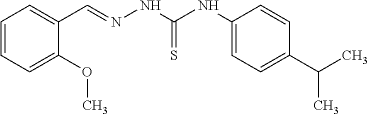 | approx 5,000 nM | |
| CB44 | 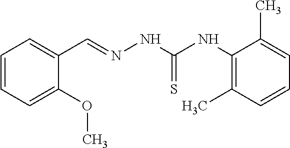 | approx 5,000 nM | |
| CB45 | 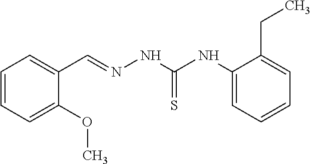 | 13 nM | |
| CB46 | 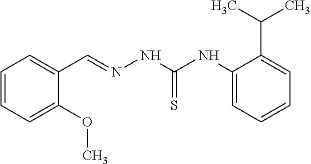 | 3 nM | |
| CB47 | 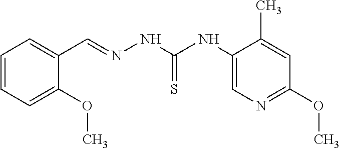 | ~300 nM | |
| CB48 | 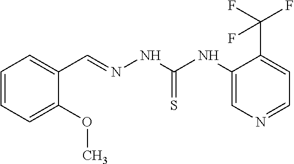 | 100-300 nM | |
| CB49 | 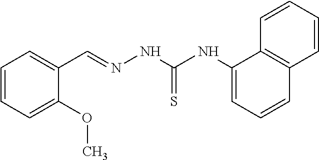 | 100-300 nM | |
| CB50 | 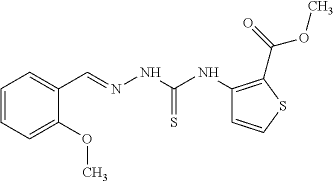 | ~3,000 nM | |
| CB51 | 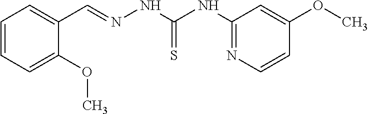 | ~10,000 nM | |
| CB52 | 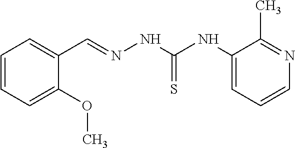 | 100-300 nM | |
| CB53 | 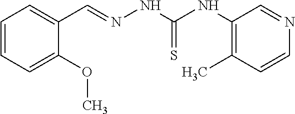 | ~10,000 nM | |
| CB54 | 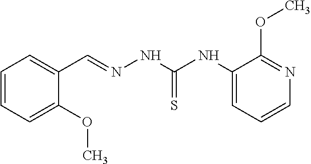 | 17 nM | |
| CB55 | 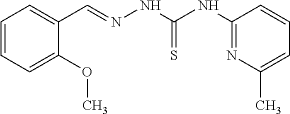 | ~1,000 nM | |
| CB56 | 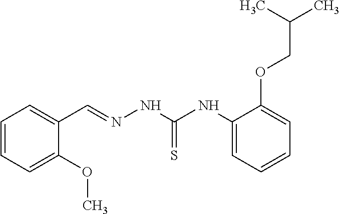 | | ~1,000 nM |
| CB57 | 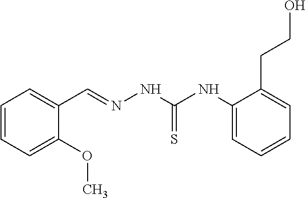 | | ~2,000 nM |
| CB58 | 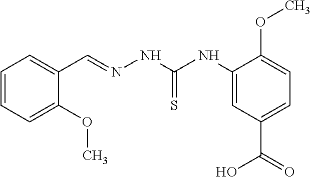 | | ~10,000 nM |
| CB59 | 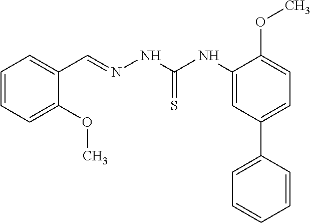 | | ~10,000 nM |
| CB60 | 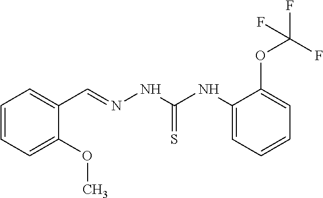 | | ~500 nM |
| CB61 | 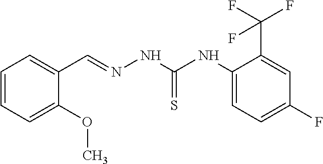 | | ~200 nM |
| CB62 | 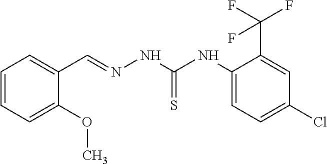 | | ~50 nM |
| CB63 | 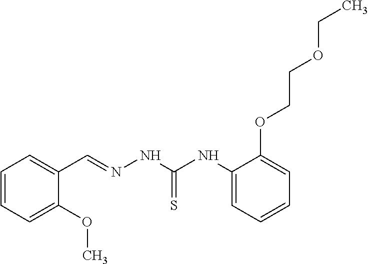 | | Approx. 2,000 nM |
| CB64 | 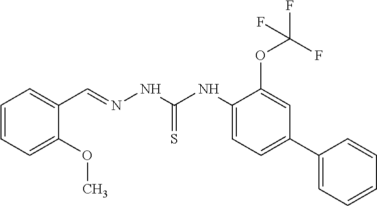 | | >10,000 nM |
| CB65 | 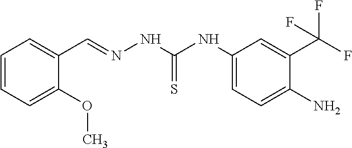 | | 100 nM |
| CB66 | 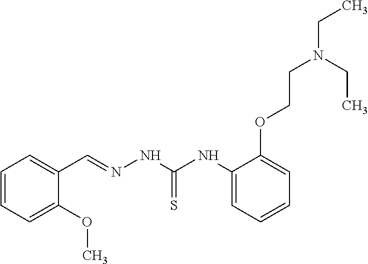 | | >10,000 nM |
| CB67 | 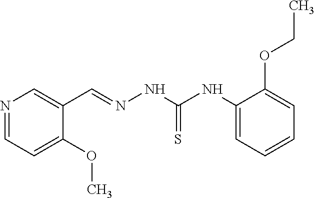 | | >3,000 nM |
| CB68 | 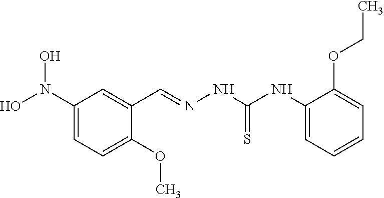 | | >10,000 nM |
| CB69 | 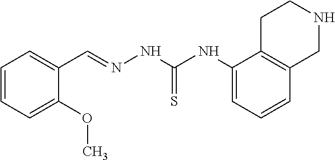 | | >10,000 nM |
| CB70 | 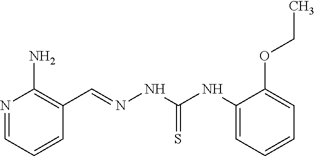 | | Approx. 1,000 nM |
| CB71 | 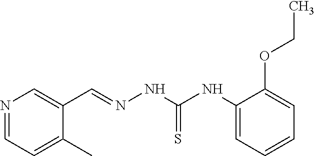 | | Approx 500 nM |
| CB72 | 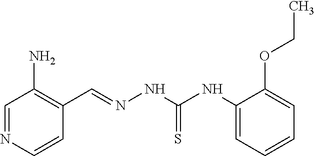 | | >10,000 nM |
| CB73 | 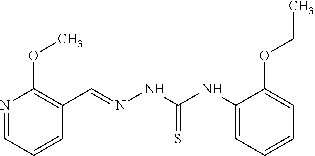 | | approx. 1,000 nM |
| CB74 | 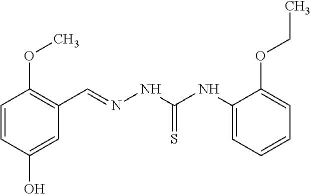 | | approx. 1,000 nM |
| CB75 | 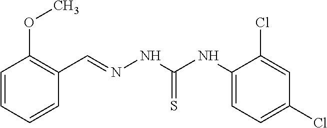 | | 20 nM |
| CB76 | 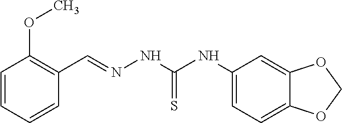 | | >10,000 nM |
| CB77 | 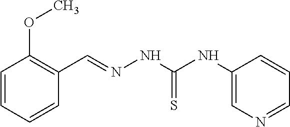 | | Approx. 2,000 nM |
| CB78 | 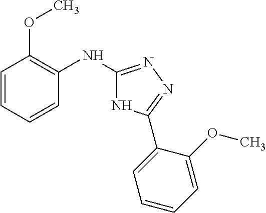 | | >10,000 nM |

1. A compound of general formula (I) or a pharmaceutically acceptable salt or solvate or thereof: 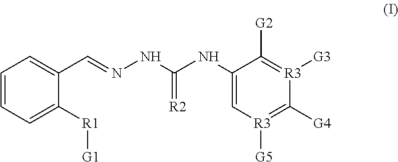
wherein:
R1 is S or O
R2 is O or NH
R3 is C or N
G₁ is H or alkyl
G_{2,3,4,5} is H, halide, alkyl, alkoxy, cyano, alkylamino, hydroxyl or fused phenyl ring;
and the carbon-nitrogen double bond is preferably in the E conformation.

2. A compound according to claim 1, wherein G₁ is H or CH₃

3. A compound according to claim 1 or claim 2, wherein R1 is S or O

4. A compound according to any one of claims 1 to 3, wherein G₂ is H, F, Cl, Br, CH₃, CF₃, Ethyl, iPropyl, O—CH₃, O—CH₂CH₃, CN, NH₂, OH or a fused phenyl ring.

5. A compound according to any one of claims 1 to 4, wherein G₃ is H, F, Cl, Br, CH₃, CF₃, Ethyl, iPropyl, O—CH₃, O—CH2CH3, CN, NH₂, OH or a fused phenyl ring

6. A compound according to any one of claims 1 to 5, wherein G₄ is H, F, Cl, Br, CH₃, CF₃, Ethyl, iPropyl, O—CH₃, O—CH₂CH₃, CN, NH₂, OH or a fused phenyl ring

7. A compound according to any one of claims 1 to 6, wherein G₄ is H, F, Cl, Br, CH₃, CF₃, Ethyl, iPropyl, O—CH₃, O—CH₂CH₃, CN, NH₂, OH or a fused phenyl ring

8. A compound selected from the group consisting of: 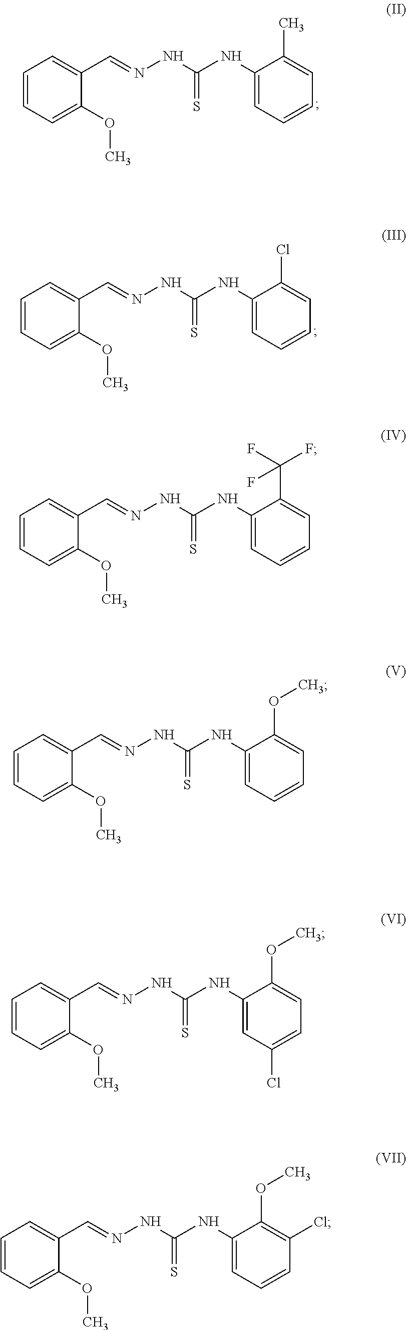 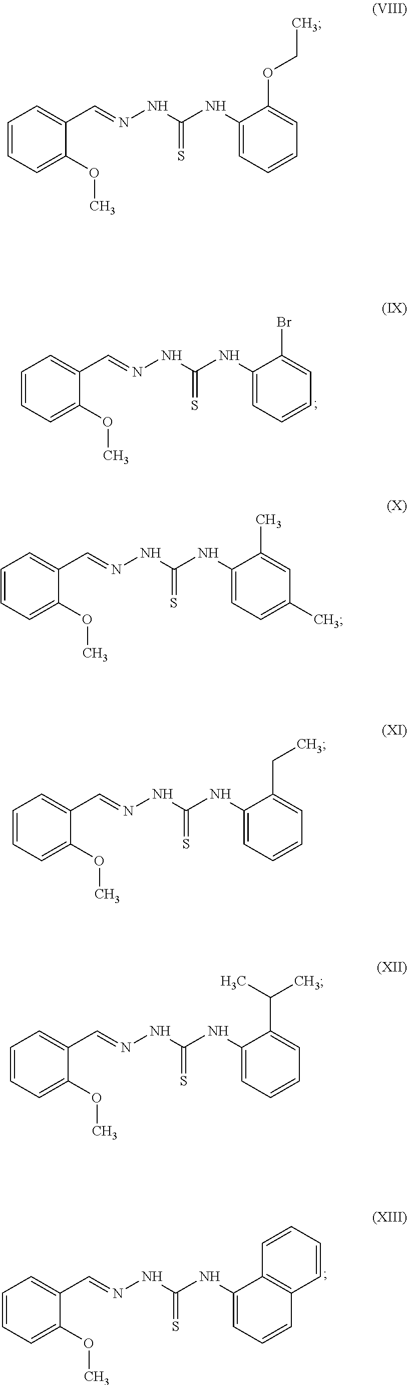 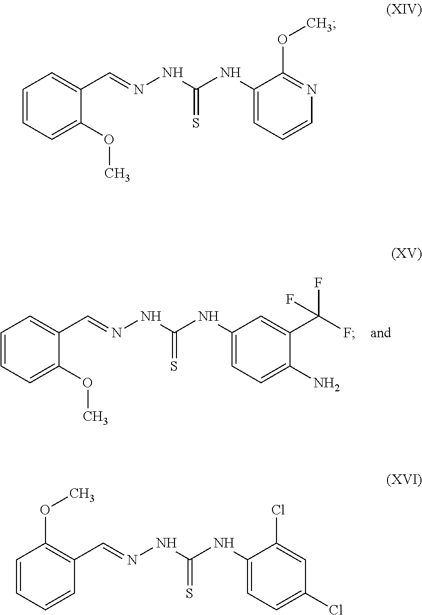
or pharmaceutically acceptable salt or solvate thereof or combination thereof.

9. A composition comprising one or more compounds according to any one of claims 1 to 8, and a pharmaceutically acceptable carrier, diluent, excipient or a mixture thereof.

10. A composition comprised of a compound according to any one of claims 1 to 9 and a polymeric material.

11. A composition according to claim 10, wherein said polymeric material is absorbable in living tissue.

12. A composition according to claim 10 or claim 11, wherein said polymeric material is selected from the group consisting of a silicon-containing polymeric material and a hydrocarbon-containing polymeric material.

13. A method treating diseases involving cell proliferation, migration, apoptosis, or adhesion, comprising administering to a human or non-human mammalian patient an effective amount of a compound according to any one of claims 1 to 8 or a composition according to any one of claims 9 to 12 or a mixture thereof.

14. A method of treating proliferative or vascular disorders, said method comprising administering to a human or non-human mammalian patient a therapeutically effective amount of a compound according to any one of claims 1 to 8, such that said proliferative disorder is treated.

15. A method according to claim 14, wherein said proliferative disorder is cancer or leukaemia.

16. A method of inhibiting microtubule cytoskeleton function, comprising contacting said microtubules with a compound according to any one of claims 1 to 8 or a composition according to any one of claims 9 to 12 or a mixture thereof, such that said microtubule cytoskeleton function is inhibited.

17. A kit comprising: a) a compound according to any one of claims 1 to 8 or a composition according to any one of claims 9 to 12 or a mixture thereof, in a unit dosage form; and b) a container means for containing said dosage form; and optionally c) with instructions for use.

18. A compound of general formula (I) or a pharmaceutically acceptable salt or solvate or thereof for use in treating diseases involving cell proliferation, migration, apoptosis, or adhesion.

19. Use of a compound of general formula (I) or a pharmaceutically acceptable salt or solvate or thereof in the manufacture of a medicament used in treating diseases involving cell proliferation, migration, apoptosis, or adhesion.

20. A compound of general formula (I) or a pharmaceutically acceptable salt or solvate or hereof: 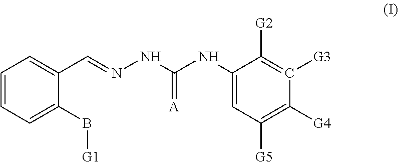
wherein:
A=S or O
B=O or N
C or N
G₁=H or alkyl
G_{2,3,4,5}=H, halide, alkyl, alkoxy, cyano, amino; hydroxyl or fused
phenyl ring
and the carbon-nitrogen double bond is the E conformation.

21. A compound according to claim 20, wherein A is H or CH3

22. A compound according to claim 20 or 21, wherein B is S or O

23. A compound according to claim 20, 21, or 22, wherein G₂ is H, F, Cl, Br, CH3, CF3, Ethyl, iPropyl, O—CH3, O—CH2CH3, CN, NH2, OH or a fused phenyl ring

24. A compound according to claim 20, 21, 22, or 23, wherein G₃ is H, F, Cl, Br, CH3, CF3, Ethyl, iPropyl, O—CH3, O—CH2CH3, CN, NH2, OH or a fused phenyl ring

25. A compound according to claims 20, 21, 22, 23, 24, wherein G₄ is H, F, Cl, Br, CH3, CF3, Ethyl, iPropyl, O—CH3, O—CH2CH3, CN, NH2, OH or a fused phenyl ring

26. A compound according to claims 20, 21, 22, 23, 24, 25, wherein G₄ is H, F, Cl, Br, CH3, CF3, Ethyl, iPropyl, O—CH3, O—CH2CH3, CN, NH2, OH or a fused phenyl ring

27. A compound selected from the group consisting of: 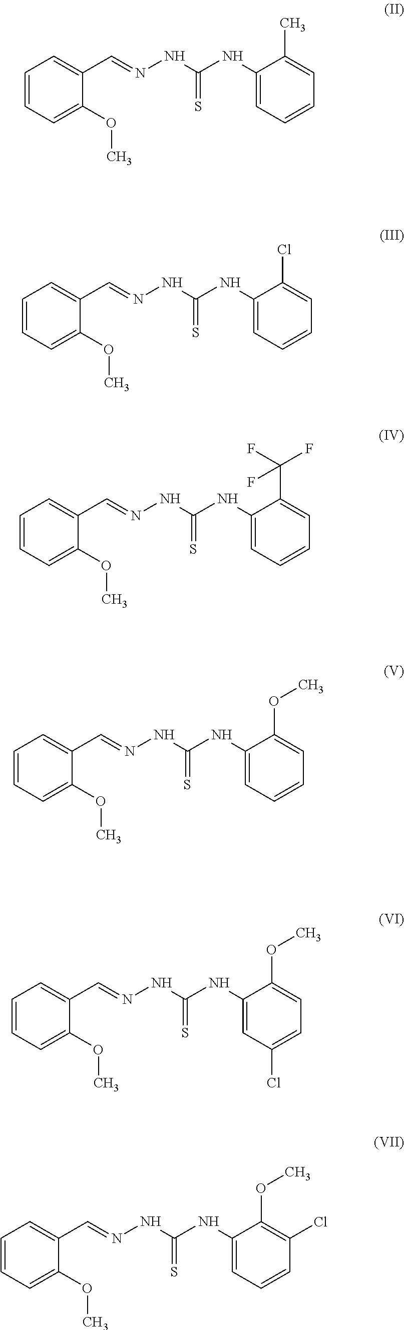 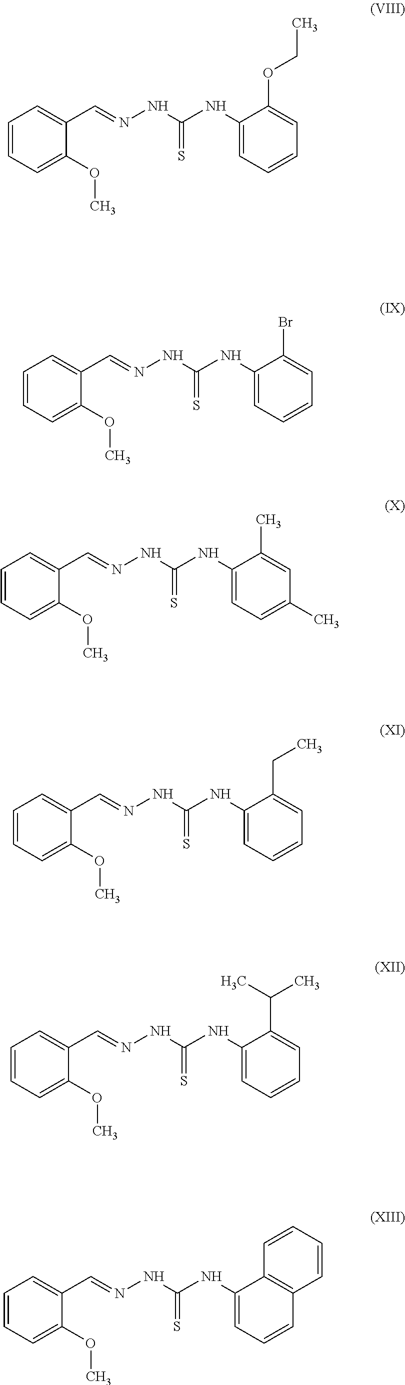 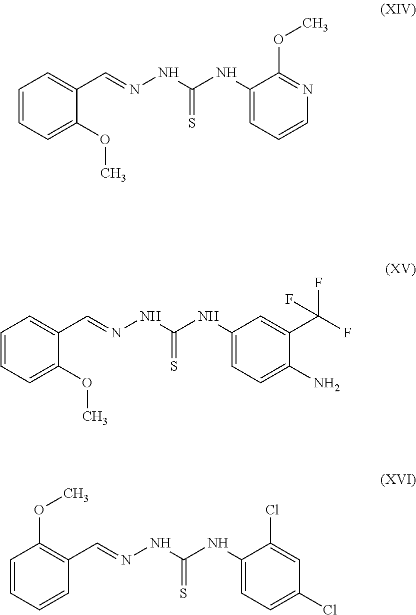
or pharmaceutically acceptable salt or solvate thereof or combination thereof.
